(19) United States
(12) Patent Application Publication
(10) Pub. No.: US 2006/0240474 A1
(43) Pub. Date: Oct. 26, 2006

(54) Peptides able to switch between two structural states and their use

(75) Inventors: Derek N Woolfson, Bristol (GB); Maya J. Pandya, Sussex (GB)

Correspondence Address: BANNER & WITCOFF, 1001 G STREET N W, SUITE 1100, WASHINGTON, DC, 20001, US

(73) Assignee: UNIVERSITY OF SUSSEX, SUSSEX (GB)

(21) Appl. No.: 10/525,388

(22) PCT Filed: Aug. 28, 2003
(86) PCT No.: PCT/GB03/03724

§ 371 Date: Jun. 8, 2006

(30) Foreign Application Priority Data
Aug. 28, 2002 (GB) 0219908.0

(51) Int. Cl. G01N 33/53; C07K 14/47

(52) U.S. Cl. 435/7.1; 530/324

(57) ABSTRACT

The present invention relates to peptides that switch between 2 structural states in response to the presence or absence of a stimulus. The present invention also relates to the use of the peptides of the present invention in an assay to detect the presence or absence of a stimulus. The present invention also relates to a method for constructing the peptides of the present invention.

The present invention relates to peptides that switch between 2 structural states in response to the presence or absence of a stimulus. The present invention also relates to the use of the peptides of the present invention in an assay to detect the presence or absence of a stimulus. The present invention also relates to a method for constructing the peptides of the present invention.

Successful protein and peptide designs require reliable rules that relate protein sequence and structure. This is reflected in the bias towards predominantly α-helical, and, in particular, coiled-coil structures in successful designs made to date, which include: four-helix bundles, parallel dimeric and trimeric coiled coils and helix-turn-helix motifs. These early α-helical designs were backed by good experimental and theoretical models for α-helical peptides, and the resulting development of rules for the folding of α-helical structures. Studies on the folding and design of β-structures are also being pursued. Recently, however, the folding of small peptides into highly populated β-structures in solution has been reported; and rules are being established that link sequence and β-structure in proteins.

Large conformational transitions within proteins, by which is meant large relative movements of, or changes in secondary structure, are increasingly being recognized as a means through which protein function (and dysfunction) is elicited and mediated. For example, in the serpins, which are suicide inhibitors of serine proteases, cleavage of the scissile bond leads to the transformation of a native loop into an internal strand of a β-sheet (Lee, Park et al., Natural Structural Biology, 3(6): 497-500, 1996) this conformational change cements interactions between the cleaved serpins and their targets. Peptides and proteins that rearrange to amyloid-like structures represent another type of transition in which β-structure is formed (Rochet and Lansbury et al., Current Opinion in Structural Biology, 10(1): 60-60, 2000) unfolded and folded polypeptides, which are not necessarily β-structured, transform to fibres rich in β-sheet. The archetypal example of conformational change involving the rearrangement of largely helical structure is the switching of influenza hemagglutinin into an active form competent for virus-host membrane fusion (Skehel and Wiley et al., Annual Review of Biochemistry, 69: 531-569, 2000). On a similar theme, the assembly of certain SNARE-protein complexes, which mediate the fusion of vesicle membranes, is inhibited by the native conformation of one component that must open prior to SNARE-complex assembly (Munson et al., Nature Structural Biology, 7(10): 894-902, 2000, Tochio et al., Science, 293(5530): 698-702, 2001). Transitions in structures that are accompanied by changes in protein oligomer state and, in turn, protein function include the release and assembly of the trimerisation region and subsequent DNA binding of certain heat shock transcription factors (Rabindran et al., Science, 259: 230-234, 1993) and the dimer-tetramer switch in IF₁, which regulates the mitochondrial ATP synthase (Cabezon et al., EMBO Journal, 20(24): 6990-6996, 2001). International patent application WO 99/11661 discloses a protein comprising an elastomeric peptide which increases in length in response to an increase in temperature; however, there is no overall change in the structure of the protein. Finally, pH changes and proximity to membranes triggers certain colicin domains to switch from water-soluble globular structures to integral-membrane proteins, which then switch between an open and closed channel state depending on an applied membrane electrical potential.

In other cases, conformational changes appear to be abnormalities, or misfolding events and result in disease. For example, in Alzheimer's dementia and prion diseases α-to-β structural transitions in peptides and proteins are implicated in the formation of amyloid fibrils and the pathogenesis of disease. Jarvet et al., (J. Am. Chem. Soc., 122, 4261-4268, 2000) discusses such structural changes in the Alzheimer peptide fragment Aβ(12-28).

The examples given above may be regarded as proteins with internal structural conflicts; that is, protein sequences with different accessible folded states and the form that is expressed depends on the prevailing conditions. This appears to run against Anfinsen's hypothesis that proteins adopt the thermodynamically most-stable state (Anfinsen et al., Science, 181: 223-230, 1973). This idea is not new, however: for example, the cross-β structure of amyloid fibres has long been considered the true energy minimum for all polypeptide chains; native influenza hemagglutinin is described as a spring-loaded, metastable conformation that is trapped awaiting a trigger to switch to the fusogenic form (Carr et al., PNAS USA, 94(26): 14306-14313, 1997); the native serpins are referred to as stressed states, cleavage of which by the targeted protease brings about a conformational transition to the relaxed state (Lee, Park et al., Nature Structural Biology 3(6): 497-500, 1996, Whisstock et al., Trends in Biochemical Sciences, 23: 63-67, 1998) and the inactive forms of certain SNAREs are referred to as “closed” conformations in which the SNARE oligomerisation motif is rendered inaccessible (Munson et al., Nature Structural Biology, 7(10): 894-902, 2000, Tochio et al., Science, 293(5530): 698-702, 2001).

One way to consider designing conformational switches is to set up a structural conflict within a peptide or protein by superimposing motifs for two different structures in a single sequence. In this way, Anfinsen's basic tenet of protein folding is not contravened, it is simply that one sequence motif will be frustrated when the alternative structure is adopted. The inventors refer to such polypeptides as having sequence and structural duality. The inventors introduced and tested this concept previously. In particular, Ciani et al. (J. Biol. Chem., 277, 10150-10155, 2002) designed peptides that switched from a α-helical structure to a β-helical structure. It was only possible to reverse the conformation of the peptides from the β structure (amyloid like fibrils) to the α-helical structure by cooling the sample, lowering its pH to pH 2 and waiting a number of days. Such a change would be impractical and, therefore, more or less useless in any embodiments of switching peptides in diagnostics, etc.

The Abstract from Japanese Patent Application JP-A-7157499 discloses a peptide that is indicated as reversibly switching between an α-helical structure and a β-sheet structure. No data is provided showing reversible switching an the sequence of the peptide is not disclosed.

Zhao et al., (Protein Science, 10, 1113-1123, 2001) discloses that the yeast adhesion protein α-agglutinin can reversibly switch from a β rich structure to a mixed α/β structure by changes in temperature or pH. Reed et al., (Biochemistry, 30, 4521-4528, 1991) discloses that peptides derived from the CD4 binding domain of gp120 switch from a α-helical structure to a β-sheet structure following changes in the polar conditions. The papers provide no information concerning obtaining or designing other switching peptides.

There is a requirement for a peptide that can reversibly switch between two different structural conformation in response to the presence or absence of a stimulus. It is also desirable that the peptide can switch between the two different structural conformations without the requirement of having to denature the protein by heating, or irreversible chemical actions.

It is also desirable to be able to provide a peptide that can be used to detect the presence of a variety of different stimuli, including pH, redox conditions, the presence of a metal ion, an antigen or a ligand. The ability to easily distinguish between the different structural conformations of the peptides is also desirable.

In a first aspect of the present invention there is provided a peptide having sequence and structural duality which can reversibly switch between a first structural form that can oligomerise and a second monomeric structural form in response to a stimulus, wherein the peptide comprises interactive motifs that interact to form a bond in response to the stimulus and thereby cause the peptide to assume the second structural form.

In a second aspect of the present invention there is provided a peptide having sequence and structural duality which can reversibly switch between a first structural form and a second structural form in response to a stimulus, wherein the peptide comprises interactive motifs that interact to form a bond in response to the stimulus and thereby cause the peptide to assume the second structural form, wherein the peptide does not switch between an a helical structure and a β sheet structure.

The peptide of the present invention (according to both the first and second aspects of the present invention) has sequence and structural duality so that the peptide can assume two different structural forms and reversibly switch between the forms in response to a stimulus. The peptide can therefore be used to detected the presence or absence of a stimulus and monitor for changes in the level of the stimulus because the peptide can reversibly switch between the two structural forms.

The peptide can switch between the different structural forms without the need to denature the protein by heating or chemical reactions, e.g. reducing the pH to pH 2. Furthermore, the switch between structural forms also occurs relatively quickly, e.g. in less than one or two hours.

The term “peptide” as used herein refers to a polymer of amino acids. The peptide can be of any length but is preferably between about 20 and 400 amino acids in length. It is further preferred that the peptide is between about 30 and 200 amino acids in length. The term does not refer to or exclude post-expression modifications of the peptide, for example, glycosylations, acetylations and phosphorylations. Included in the definition are peptides containing one or more analogues of an amino acid, including unnatural amino acids.

The term “sequence and structural duality” means that the peptide has a sequence that is compatible with 2 different structures. Generally, the peptide has 2 different superimposed sequence and structural motifs enabling the peptide to assume two different structures, namely a first and a second sequence and structural motif. Preferably the first sequence motif of the peptide is a coiled-coil motif for a parallel coiled-coil dimer structure. It is also preferred that the second sequence motif of the peptide is a coiled-coil motif for an anti-parallel coiled-coil monomer structure.

The term “reversibly switch” is used to indicate that the peptide can reversibly switch between the first and second structural forms. Therefore the peptide can switch from the first structural form to the second structural form and back again. As indicated above the majority of prior art peptides that are capable of switching structure can only switch from a first structure to a second structure but not back again. Such prior art peptides cannot be reused and cannot be used to monitor changes in the level of a stimulus because once the peptide assumes the second structure it is no longer responsive to the stimulus.

The first and second structural forms of the peptide according to the present invention can be any structural forms provided they are sufficiently different from each other to enable one skilled in the art to easily determine if the peptide is in the first structural form or the second structural form, and preferably to measure the ratio between the first and second structural forms of the peptide. In the first aspect of the present invention, and preferably in the second aspect of the invention, the first structural form of the peptide oligomerises and the second structural form of the peptide is a monomer. This is advantagous because it is relatively easy to distinguish between an oligomer and a monomer. Suitable methods of determining if the peptide is in the first or second structural form include circular dichroism spectroscopy and non-denaturing PAGE. Split proteins and enzymes may also be used to detect the confirmational change of the protein (see Ghosh et al., J. Amer. Chem. Soc., 122, 5658-9, 2000; Johnsson et al., PNAS USA, 91, 10340-4, 1994, and Hocker et al., Nat. Struct. Biol., 8, 32-36, 2001).

Preferably, the first structural form is a continuous helical structure. In the first aspect of the present invention, and preferably in the second aspect of the present invention, the first structural form can oligomerise. The peptide in the first structural form may form a heteroligomer or a homooligmer. It is particularly preferred that the peptide in the first structural form is a parallel coiled-coil homodimer.

Preferably, the second structural form is a hairpin structure. It is particularly preferred that the peptide in the second structural form is an anti-parallel coiled-coil monomer.

The term “interactive motifs” as used herein means any motifs which can interact with each other to form a covalent or non-covalent bond or bonds in response to a stimulus. Preferably the interactive motifs interact to form a non-covalent bond or bonds. By the interactive motifs forming a bond the peptide of the present invention is caused to assume the second structural form.

The interactive motifs may be adjacent to each other and positioned as a single unit in the peptide. For example, where the interactive motifs are part of a metal binding site, the metal binding site, or part of the metal binding site comprising both interactive motifs, may be positioned with the peptide as a single unit. Alternatively, the interactive motifs can be in separate parts of the peptide. The interactive motifs can be at any position in the peptide provided they can interact to form a bond or bonds. Preferably, when the interactive motifs are present as a single unit, they are positioned towards the middle of the peptide. Preferably, when the interactive motifs are in separate parts of the peptide, they are at or near each end of the peptide. There may be a number of interactive motifs which all interact to form the bond or bonds. Preferably there are only two interactive motifs and they are preferably positioned at each end of the peptide.

A number or specific examples of interactive motifs are discussed below.

The interactive motifs may be cysteine residues that form a di-sulphide linkage in response to the stimulus of an oxidising environment.

The interactive motifs may comprise parts of a metal binding site that form a non-covalent linkage in response to the stimulus of the presence of the corresponding metal ion. Suitable metal binding sites are known to those skilled in the art, including zinc fingers and lanthanide-binding motifs. A part of the metal binding site can be incorporated into one part the peptide of the present invention and a second part of the metal binding site can be incorporated into a second part of the peptide of the present invention, wherein when the metal ion is present, the parts of the metal binding site interact via the metal ion to form a bond or bonds.

The interactive motifs may comprise parts of an antigen binding site of an antibody molecule which form a non-covalent linkage in response to the stimulus of the presence of the corresponding antigen. Those skilled in the art have used single chain Fv fragments of antibody molecules to successful bind antigen. Furthermore, people skilled in the art have incorporated the variable regions of antibody molecules into proteins. Accordingly, one skilled in the art could easily incorporate antigen binding regions of an antibody molecule into the peptide of the present invention using known techniques. Suitable framework regions of the antibody molecule can be included in order to ensure that the antigen binding regions are correctly orientated. As antibodies can be generated to almost any antigen, the peptide of the present invention can be used to detect the presence of almost any antigen. As indicated above with respect to the metal binding sites, parts of the antigen binding site can be incorporated into the peptide of the present invention at different sites, wherein when the antigen is present the parts of the antigen binding site interact to form a bond or bonds.

The interactive motifs may comprise parts of a ligand binding site of a receptor that form a non-covalent linkage in response to the stimulus of the presence of the corresponding ligand. Numerous ligand binding sites are known to those skilled in the art and the necessary parts of the ligand binding sites could be incorporated into the peptide of the present invention. As indicated above, parts of the ligand binding site can be incorporated into the peptide of the present invention at different sites, wherein when the ligand is present the parts the ligand binding site interact to form a bond or bonds.

The stimulus that causes the peptide of the present invention to switch conformation will, as indicated above, depend on the interactive motifs in the peptide. The stimulus may be the presence or absence of a particular redox state, a metal ion, an antigen or a ligand.

In a particularly preferred embodiment of the present invention the peptide according to the present invention has the following sequence:
X¹-(Y)ₙ-(abcdefg)ₘ-abcdef((Z)ₚ)g-(abcdefg)ₘ-abcde-(Y)ₙ-X²

wherein:

X¹ and X² are motifs capable of interacting to form a bond;

Y is any amino acid capable of acting as a linker;

ₙ is independently selected form 0 to 20;

abcdefg is a heptad sequence motif;

^{m} is 1 to 20;

abcde is the first 5 residues of a heptad sequence motif;

Z is an amino acid that is compatible with the 2 structural forms of the peptide; and

ₚ is 1 to 6.

X¹ and X² can be any motifs which are capable of interacting to form a bond or bonds in response to the presence or absence or a stimulus. As indicated above, the bond or bonds may be covalent or non-covalent. X¹ and X₂ are can represent any interactive motif as defined above. Preferably, X¹ and X² are amino acids capable of forming a disulphide link. It is particularly preferred that X¹ and X² are cysteine.

Y is preferably independently selected from glycine, serine or β-alanine. It is particularly preferred that Y is glycine and ₙ is 3.

The term “heptad repeat” refers to any seven amino acid repeat which is capable of forming a helical coiled-coil structure. Suitable heptad repeat sequences are well known to those skilled in the art.

Preferably the d of the heptad sequence is leucine. It is further preferred that the a of the heptad sequence is independently selected from isoleucine, valine, lysine, asparagine and arginine, provided a is lysine, asparagine or arginine in only 1 in every 4 heptad sequences of the peptide. It is particularly preferred that the a of the heptad sequence is isoleucine or lysine provided a is only lysine in one of the heptad sequences of the peptide.

It has been found that by restricting the a and d residues as indicated above, heptad repeats are formed that code particularly well for two-helix coiled-coil structures.

It is also preferred that the g and e residues of the heptad repeat are oppositely charged and the polarity of the g and e are reversed in the C-terminal half of the peptide compared to the N-terminal half of the peptide. It has been found that by restricting the g and e residues as indicated above, the heptad is compatible with forming a parallel coiled-coil dimeric structure and an anti-parallel coiled-coil monomeric structure.

m is preferably between 1 and 5, most preferably m is 1 or 2.

Z may be a flexible amino acid, namely an amino acid that is capable, in an appropriate sequence context, of being flexible enough to accomodate the 2 structural forms. In particular, the group (Z)ₚ is capable of forming part of coiled coil structure and of forming the hairpin structure of an anti-parallel coiled coil monomer. Z is preferably independently selected from glycine, alanine and glutamine. It is also preferred that ₚ is 4. It is particularly preferred that (Z)ₚ is Ala-Lys-Gln-Ala or Ala-Ala-Gln-Ala.

In specific embodiments of the present invention, the peptide of the present invention has the following sequence:

| | |
|---|---|
| CGGEIRALKYEIARLKQAKQAKIRALEQKIAALEGGC | (CSP-3); |
| CGGEIRALKYEIARLKQAAQAKIRALEQKIAALEGGC | (CSP-6); |
| or | |
| CGGEIRALKYEIARLKQAAQAKKRALEQKIAALEGGC | (CSP-7). |

The names CSP-3, CSP-6 and CSP-7 derive from the orginal design process and have been included to tally with the Figures and experimental section.

As will be apparent to those skilled in the art modifications of the sequence of the peptides of the present invention can be made to alter the peptides characteristics. For example, modifications can be made to the peptide so that it requires a greater stimulus to switch conformation, thereby enabling the peptide to be used to detect on high level of the stimulus. Other modification, well known to those skilled in the art, can be made to the peptide to increase its solubility, etc.

The present invention also provides the use of the peptide according to any one of the preceding claims for determining the presence or absence of a stimulus.

As indicated above, the peptide of the present invention can be used to determine the presence or absence of a stimulus. The stimulus to be detected will depend on the particular peptide be used as discussed above.

The present invention provides a method of detecting a stimulus comprising incubating a peptide of the present invention with a test solution, and determining if the peptide has the first structural form or the second structural form in the test solution, wherein if the peptide assumes the second structural form, the test solution contains the stimulus.

The change in the structure of the peptide can be measured using any technique. Suitable techniques include circular dichroism spectroscopy, fluorescence quenching, Fluorescence Resonance Energy Transfer (FRET) and non-denaturing PAGE (particularly where the peptide oligomerises in only one structural form). Split proteins and enzymes may also be used to detect the conformational change of the protein (see Ghosh et al., J. Amer. Chem. Soc., 122, 5658-9, 2000; Johnsson et al., PNAS USA, 91, 10340-4, 1994, and Hocker et al., Nat. Struct. Biol., 8, 32-36, 2001). In the case of FRET, donor and acceptor fluorophores can be added to parts of the peptide so that they are only brought into proximity with each other in one of the conformational states.

The present invention also provides a method for constructing a peptide that has sequence and structural duality, which can reversibly switch between a first and a second structural form in response to a stimulus, comprising:
(i) designing and producing a peptide using sequence rules for the first and second structural forms, wherein the sequence rules are superimposed in the peptide; and
(ii) providing interactive motifs in the peptide that form a bond in response to a stimulus, wherein the bond stabilises the second structural form, and wherein in the absence of the bond the second structural form is not stabilised and the peptide preferentially forms the first structural form.

The first and second structural forms can be any structural forms. Rules for various structural forms are well know to those skilled in the art. For example, rules for the parallel coiled-coil dimer, the coiled-coil anti-parallel monomer, the β-sheet, coiled-coil trimers and tetramers, four helix bundles, leucine rich repeats, zinc fingers, etc. are well known (Kajara et al., Protein Science, 11, 1082-1090; and Woolfson, Current Opinion in Structural Biology, 11, 464-471, 2001).

Preferably the method is used to construct the peptide according to the first or second aspects of the present invention. The terms used in the method described above have the same meaning as that indicated earlier in the description.

The term “sequence rules are superimposed” as used herein means that the sequence of the peptide satisfies the rules for both the first and second structural forms. This enables the peptide to adopt the first and second structural forms.

The term “stabilise the second structural form” means that the bond formed by the interactive motifs in response to the stimulus specifically stabilises the second structural form and thereby allows the peptide to assume the second structural form. In the absence of the bond, the peptide preferentially assumes the first structural form. The sequence of the peptide is therefore designed to ensure that the first structural form is preferentially formed in the absence of the bond. This can be achieved by ensuring that the sequence of the peptide satisfys the rules of the first structural form to a greater degree that rules of the second structural form. However, on forming the bond, the structure of the peptide is stabilised in an orientation that favours the formation of the second structural form. The peptide has a structural conflict and the presence or absence of the bond dictates which confirmation the peptide assumes.

The present invention is now described by way of example only with reference to the following drawings.

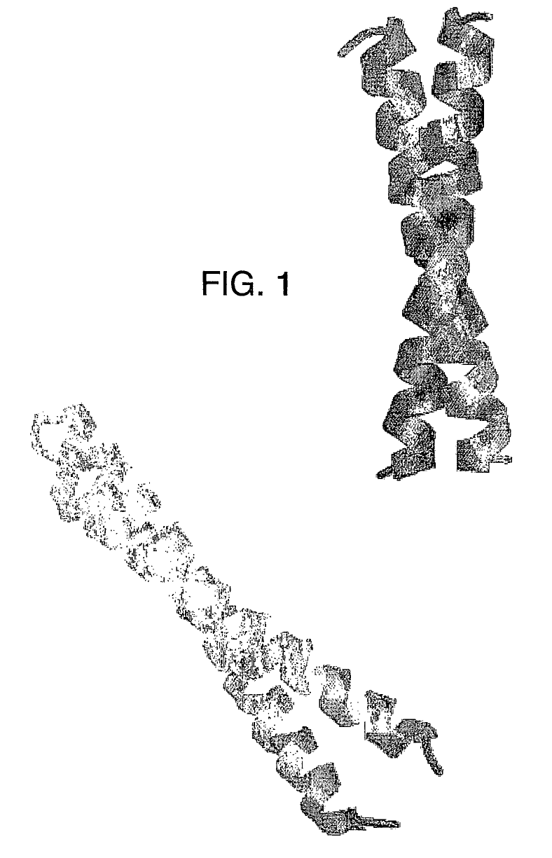

FIG. 1 shows the ribbon structures of the leucine zipper (2zta, a dimeric coiled coil) and the helical arm from the seryl tRNA synthase (1ser, an anti-parallel, two-stranded coiled coil).

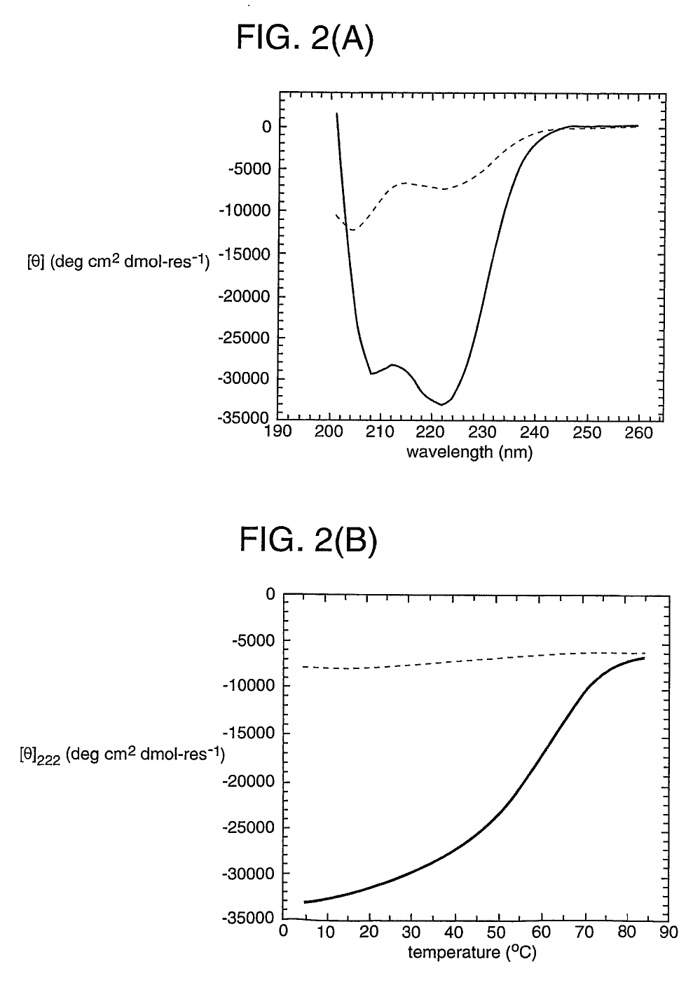

FIG. 2 shows CD spectra and thermal unfolding curves for CSP-1 (panels A and B), and CSP-3 (panels C & D) in the reduced and alkylated (solid lines) and intra-molecularly oxidised forms (broken lines).

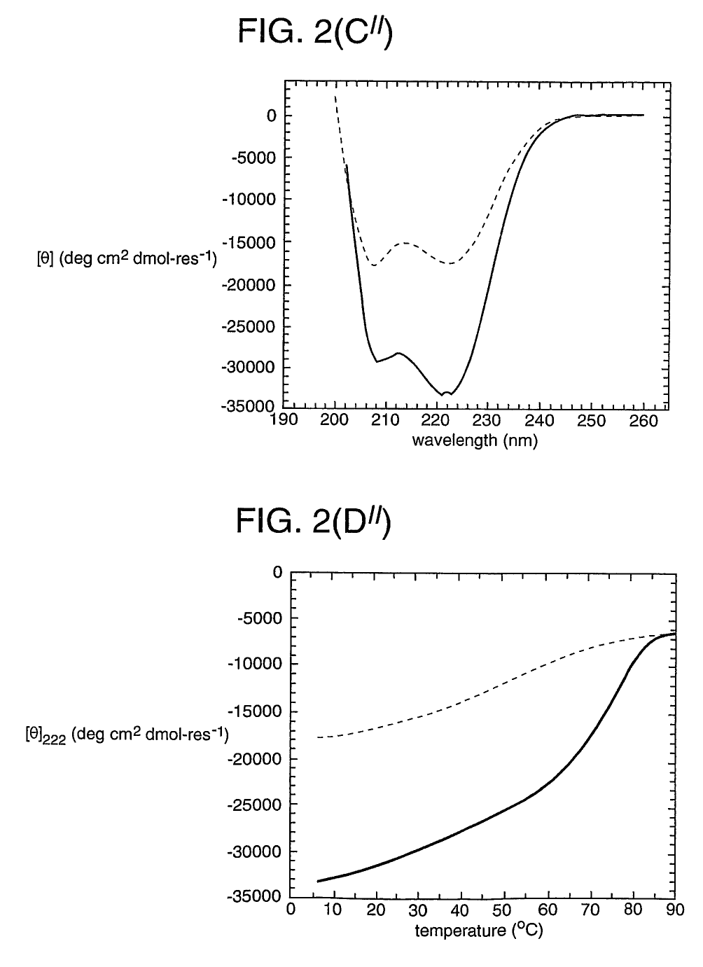

FIG. 3 shows summaries of the helicities and thermal stabilities of the reduced and oxidised variants of the CSP peptides through the design process. A. Helicities measured by the absolute value of the CD signal at 222 nm. B. Stabilities measured as the midpoints of the thermal unfolding curves (T_{M}). Crosses and solid lines are for the reduced and alkylated peptides, circles and broken lines are for the oxidised peptides.

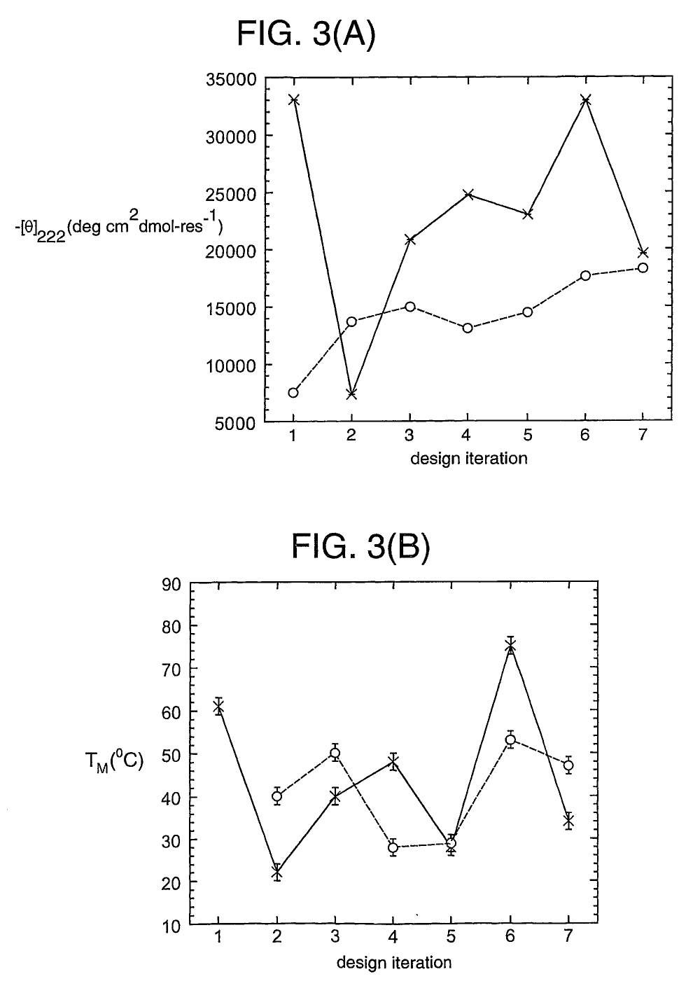

FIG. 4 is a graph showing the time taken for reduction to complete in presence of dithioerythritol (DTE) and β-mercaptoethanol (βME).

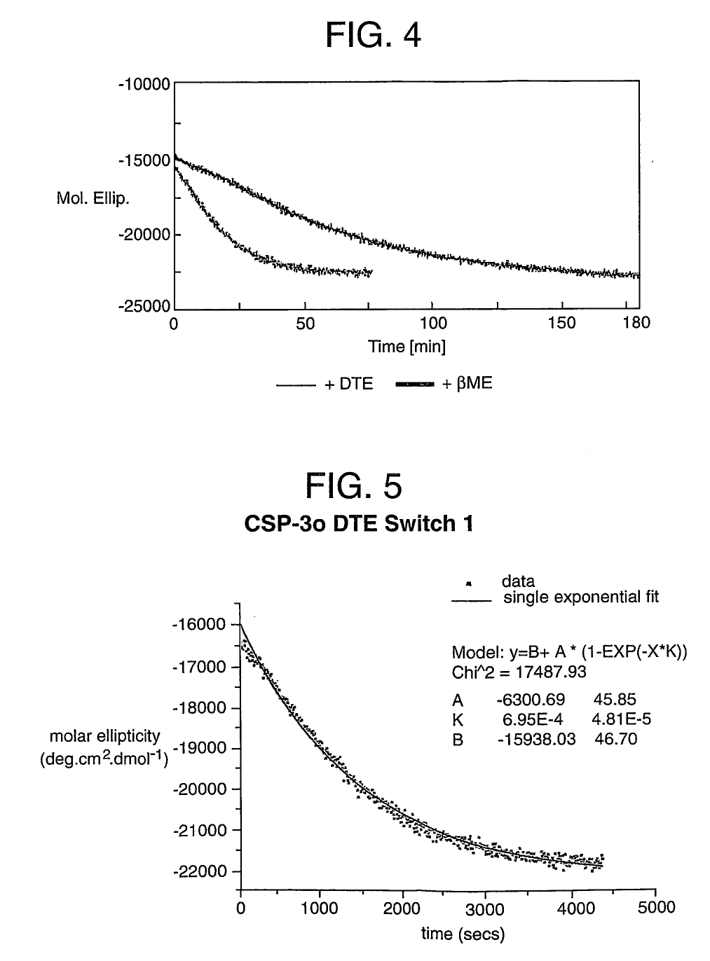

FIG. 5 is a graph showing the conformational switch of peptide CSP-3 in the presence of DTE.

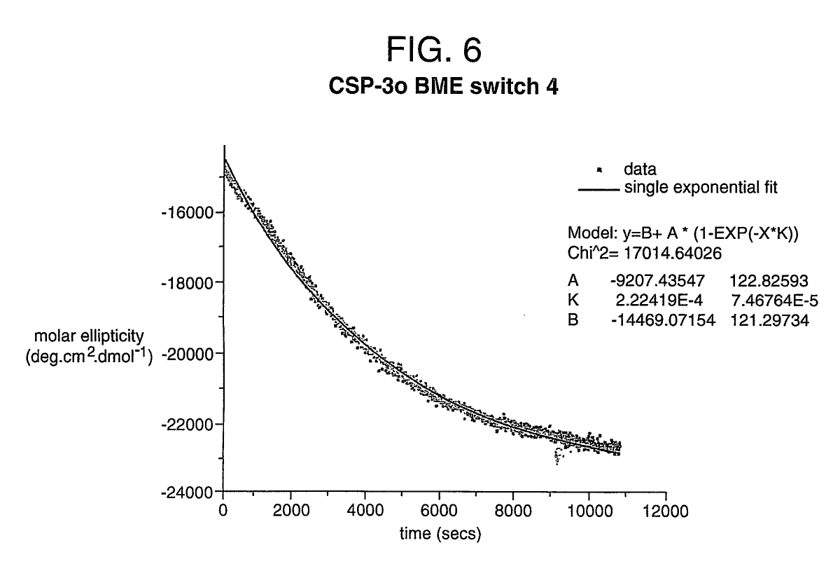

FIG. 6 is a graph showing the conformational switch of peptide CSP-3 in the presence of βME.

### EXAMPLES

The designs were based on heptad and related sequence motifs characteristic of two-helix coiled coils. The terminal residues were made cysteine to allow the two different sequence motifs to be expressed in different redox states. The inventors aimed to promote a parallel coiled-coil dimer (leucine zipper) in the reduced state, and a monomeric antiparallel, two-helix coiled coil (helical hairpin) in the intra-molecularly oxidised state. Starting with a template for a canonical leucine zipper, features were introduced to promote the alternate helical-hairpin state. The introduction of a four-residue insert to make a central non-canonical eleven-residue unit was important to obtain successful designs; the insert provided flexibility consistent with the helical hairpin, whilst remaining compatible with the coiled-coil dimer. Through this process stable structures with characteristics consistent with the target structures under the different conditions have been engineered.

Materials and Methods

Peptide Synthesis.

Peptides were made on a Pioneer Peptide Synthesis System (Perseptive Biosystems) using standard Fmoc chemistry. They were purified by reversed-phase HPLC and their identities confirmed by MALDI-TOF mass spectrometry. Purified peptides were stored at pH 2, −20° C., and concentrations were estimated by UV absorption at 280 nm (e=1490 M⁻¹ cm⁻¹). Oxidised peptides were prepared by agitation of a 100 mM peptide solution at room temperature overnight in 0.1 M Tris-HCl pH 8.5 containing 6 M guanidine hydrochloride. Alkylated peptides were prepared by (1) incubation of a 1 mM peptide solution at 40° C. for 1 h in 0.6 M Tris-HCl pH 8.6 containing 1.25% (v/v) 2-mercaptoethanol, 8 M urea, 5 mM EDTA; (2) mixing the resulting reduced peptide solutions with 0.75 ml fresh 0.36 M iodoacetamide solution and incubating at room temperature for 15 min in the dark; and (3) dialysis against water. Chemical modification of cysteines was confirmed by MALDI-TOF mass spectrometry.

Analytical Ultracentrifugation.

Sedimentation equilibrium experiments were conducted at 5° C. in a Beckman-Optima XL-I analytical ultracentrifuge fitted with an An-60 Ti rotor. A 100 ml peptide solution at 100-500 mM in 25 mM K phosphate buffer pH 7 containing 0.1 M KCl was equilibrated for 48 h at speeds of 40 000, 47 500/50 000 and 55 000/60 000 rpm. Data were fitted simultaneously assuming either a single ideal species or a monomer-dimer equilibrium and fixed monomer molecular weight, using routines in the Beckman-Optima XL-A/XLI-I data analysis software (v4.0). The molecular weights (Table 2) and partial specific volumes (range 0.7417 to 0.7461) of the peptides were calculated from their amino-acid sequences, and the viscosity of the buffer at 5° C. was taken to be 1.008 mg ml⁻¹.

Circular dichroism spectroscopy. CD measurements were made using a JASCO J-715 spectropolarimeter fitted with a Peltier temperature controller. Peptide solutions were prepared in 25 mM potassium phosphate buffer at pH 7 and were examined in 1 mm or 1 cm quartz cuvettes (with stirring). Spectra were recorded at 5° C. using 1 nm intervals, a 1 nm bandwidth and 4 sec response time. Thermal unfolding curves were recorded through 1° C. min⁻¹ ramps using a 2 nm bandwidth, averaging the signal for 16 s every 1° C. intervals. After baseline correction, ellipticities in mdeg were converted to molar ellipticities (deg cm² dmol-res-⁻¹) by normalising for the concentration of peptide bonds.

The goal of this study was to design a peptide the conformation of which could be switched by simple chemical modification. Our target conformations were the parallel and anti-parallel coiled coils (see FIG. 1), and the trigger in this specific example for the switch was an intra-molecular disulphide bridge: cysteine residues placed at the termini of the sequence could either be left reduced to favour a parallel coiled-coil dimer, or oxidised intra-molecularly to form a hairpin and favour the anti-parallel structure. Whilst good rules are available that allow confident rational designs of parallel coiled-coil peptides (particularly leucine zippers) (Harbury et al., Science, 262(5138): 1401-1407, 1993, Woolfson and Alber et al., Protein. Sci, 4(8) 1596-1607, 1995, Pandya, Spooner et al., Biochemistry, 39(30: 8728-8734, 2000, Ciani Hutchinson et al., Journal of Biological Chemistry, 277(12): 10150-10155, 2002) similar rules for anti-parallel coiled coils are only just being explored (Oakley et al., Current Opinion in Structural Biology, 11(4): 450-457, 2001, Walshaw et al., Journal of Molecular Biology, 307(5): 1427-1450, 2001). Because of this, the approach in the design of a switch was iterative: a parent peptide, Coiled-coil-Switch Peptide-1 (CSP-1), was designed as a canonical dimeric leucine zipper; small changes were then made sequentially to stabilise the anti-parallel form, whilst minimising the destabilisation of the parallel (leucine-zipper) state. Various iterations were characterised in solution using circular dichroism (CD) spectroscopy and analytical ultracentrifugation (AUC). In this way it is was possible to incorporate and test modifications designed to favour one state or the other and in a directed manner arrive at the design. This process culminated in designs that have characteristics fully compatible with the desired target structures in the appropriate states; namely, peptides that in the reduced and oxidised states formed dimers and monomers, respectively and switched between the dimeric and monomeric forms depending on the redox state. The peptides were predominantly helical; and they had sigmoidal thermal unfolding transitions consistent with the formation of unique, co-operatively folded species.

Results and Discussion

Design Principles:

The hydrophobic cores of the coiled coils are made up in layers. The usual heptad sequence motif, abcdefg, of the coiled coil contributes every 1^{st} and 4^{th} site (a and d) to the core. In parallel, homodimeric coiled coils—such as the targeted leucine-zipper structure—each layer comprises a mirrored pair of side chains from each strand; i.e. both a, or both d. The choice of residues at these sites largely dictates oligomer-state selection (Harbury et al., Science, 262(5138): 1401-1407, 1993; Woolfson et al., Protein Sci, 4(8) 1596-1607, 1995; Walshaw et al., Journal of Molecular Biology, 307(5): 1427-1450, 2001). Therefore, CSP-1 was designed as a canonical leucine zipper with all of its d sites Leu, and all but one of the a sites Ile, Table 1; dimers, but not with higher-order oligomers (Woolfson et al., Protein Sci, 4(8) 1596-1607, 1995; Gonzalez et al., Nature Struct. Biol, 3(12): 1011-1018, 1996).

Sequence-to-structure relationships are less well established for antiparallel two-stranded coiled coils. In addition, in these structures a:d and d:a pairs align alternately in the core (Oakley et al., Current Opinion in Structural Biology, 3(10) 658-667, 2001). The distinction between a:d and d:a pairs is simply that the first named position indicates the residues that appears first in the linear sequence. Nonetheless, the combination of Leu at d and predominantly Ile at a appears to be compatible with such structures (Walshaw et al., Journal of Molecular Biology, 307(5): 1427-1450, 2001). The a:d alignment had clear consequences for the initial design: using a canonical heptad repeat, the turn in the oxidised form of CSP-1 could coincide with abcd or defga. To give more flexibility, the latter was used (see Table 1), which gave alternating d:a and a:d layers emanating from the turn. We aimed to achieve this register by appropriate turn design and placement of the Cys residues at the termini. For CSP-1, the turn designed built upon the Lys at the central a site: to destabilise helical structure in this region further the local sequence was made basic (see Table 1).

Inter-helix charge-charge interactions were also incorporated in the peptide designs. In parallel structures, g and e residues of successive heptad repeats are brought close in space and flank the core residues. On this basis, many designs have used oppositely charged g:e pairs to achieve specific helix-helix interactions (O'Shea et al., Cell, 68(4): 699-708, 1992; O'Shea et al., Current Biology, 3(10): 658-667, 1993; Vinson et al., Genes Dev, 7(6) 1047-58, 1993; Kohn et al, Journal of Molecular Biology, 283: 993-1012, 1998) four such g:e pairs were included in the CSP-1 design. In anti-parallel coiled coils, however, g:g and e:e pairs flank the core (Monera et al., Journal of Biological Chemical Society, 268(26): 19218-19227, 1993; McClain et al., Journal of American Chemical Society, 123(13): 3151-3152, 2001; Oakley et al., Current Opinion in Structural Biology, 3(10): 658-667, 2001). To allow for this and to promote favourable inter-helix charge-charge interactions in both target structures, the polarity of g:e pairs between the N and C-terminal ends of CSP-1 was swapped; i.e., from the N— to the C-terminus the g:e pairs read Glu:Lys, Glu:Lys, Lys:Glu, Lys:Glu.

Finally, for each peptide the coiled-coil sequence was flanked by Gly-Gly-Cys tripeptides (see Table 1), to allow their termini to be linked intra-molecularly by a disulphide bond and so promote the anti-parallel state.

The names of the peptides were derived as follows: all were termed CSP for Coiled-coil Switch Peptide; all were given a number, CSP-#, to indicate the design iteration; all were suffixed by either “r”, or “o” to indicate whether they were studied with the terminal Cys residues reduced-and-alkylated, or oxidised as an intra-molecular disulphide bridge, respectively.

Characterisation of the Parent Design CSP-1:

The parallel coiled-coiled template design, CSP-1r, was confirmed as follows: firstly, the free Cys residues of CSP-1 were alkylated to prevent oxidation during experiments. At pH7 and 100 μM peptide concentration, CSP-1r was almost fully helical (90% as judged by circular dichroism (CD) spectroscopy (see FIG. 2A). Thermal unfolding of peptide followed by CD spectroscopy was reversible and gave a sigmoidal curve indicative of a co-operative transition (see FIG. 2B). As expected for an oligomerising system, unfolding was concentration-dependent; the transition midpoint, T_{M}, was 50° C. for a 10 mM sample (data not shown) and increased to 62° C. at 100 mM peptide. Sedimentation equilibrium analysis of CSP-1r using analytical ultracentrifugation (AUC) at pH 7 and 5° C. revealed a monomer-dimer equilibrium (see Table 2), wherein a weighted fit of the data assuming a single ideal species gave a molecular weight of 6885 compared with the monomer Mᵣ 3761. A fit to a monomer-dimer model gave a dissociation constant of 33 mM (see Table 2). These data are all consistent with CSP-1r forming a stable coiled-coil dimer as designed.

The CD and AUC measurements with the reduced (but not alkylated) and the alkylated versions of the peptide compared well (data not shown).

Intra-molecular oxidation of the Cys residues to give the disulphide-cross-linked hairpin, CSP-1o, was confirmed by mass spectrometry and sedimentation equilibrium analysis. The weighted fit of the sedimentation equilibrium data assuming a single ideal species gave a molecular weight of 3260 compared with the monomer Mᵣ 3643 (see Table 2). CD measurements indicated limited, partial folding for a 100 mM sample of CSP-1o, consistent with only 20% helix (see FIG. 2A). This signal changed little upon heating (see FIG. 3b) consistent with little, or at best ephemeral structure, and this behaviour did not change with peptide concentration. These data indicate that although CSP-1o is monomeric as designed, the helical structure in the peptide is low and ephemeral; i.e., CSP-1o does not form a good helical hairpin as desired.

Redesigning the Turn Region:

One possibility for the poor folding of CSP-1o is that the turn region is too tight. One way to provide more freedom is simply to increase the loop length (Efimov et al., Protein Engineering, 4(3), 245-250, 1991). However, increased loop lengths tend to destabilise proteins (Nagi et al., Folding & Design, 2(1): 67-75, 1997; Viguera et al., Nature Structural Biology, 4(11): 939-946, 1997). Furthermore, in the present peptides, an insertion had also to be compatible with the parallel two-stranded coiled-coil target. Therefore, the inventors chose to add four residues in the middle of the CSP-1 sequence. Regarding the fully helical dimer target, this potentially added an additional helical turn, and introduced an eleven-residue (hendecad) repeat in place of the central heptad of CSP-1. Multi-stranded, parallel coiled coils are known to tolerate such hendecad inserts (Lupas et al., TIBS, 21: 375-382, 1996; Brown et al., Proteins-Structure Function and Genetics, 26(2): 134-145, 1996; Hicks et al., Folding & Design, 2(3):149-158, 1997; Burkhard et al., Trends in Cell Biology, 11(2): 82-88, 2001). Indeed, recently the inventors have determined that for inserts of between 1 and 6 residues in otherwise heptad-based coiled coils, 4-residues inserts are the least destabilising (Hicks et al., J. Struct. Biol., 137, 73-81, 2002). Initially, in CSP-2, we tested this idea with the addition of tetraglycine after the f position of the second heptad of CSP-1, otherwise the sequence was unchanged (see Table 1).

To test if the tetraglycine insertion had improved the helical-hairpin design, the inventors made and characterised the disulphide-crosslinked version, CSP-2o. Sedimentation equilibrium analysis indicated a monomeric species in solution with an effective molecular weight very close to that expected for the peptide (see Table 2). Under the same buffer conditions used for CSP-1o, CD spectra of CSP-2o indicated approximately double the helical signal ([θ]₂₂₂) of the parent peptide FIG. 3A; from this it was estimated that CSP-2o was ˜40% helical. Furthermore, thermal unfolding of CSP-2o gave a sigmoidal transition with a midpoint of 40±2° C. (see FIG. 3B). Thus, the tetraglycine insert improved the structure and stability of helical-hairpin state as desired.

However, the reduced form of the peptide, CSP-2r, was only partially folded as judged from CD spectra (consistent with only ˜20% helix) (see FIG. 3A). Nonetheless, the thermal unfolding transition showed the latter part of a sigmoidal curve and a T_{M} of 22±2° C. (see FIG. 3B). However, sedimentation equilibrium analysis showed that the tetraglycine insertion considerably hampered dimerisation even at 5° C. The data fitted best assuming a single ideal species, and returned a molecular weight close to that expected for monomer (see Table 2). Fits to monomer-dimer were poor and gave weak dissociation constants in the 10-100 mM range.

Thus, in CSP-2 the helical-hairpin form was stabilised, but this was at the expense of the helical-dimer state. Presumably, tetraglycine favours hairpin formation because it breaks the contiguous helical stretch, but, by the same token, it destabilises the helical dimer because of its flexibility and low helix propensity. Therefore, the next step of the design requires improving the stability of the helical dimer, whilst keeping sufficient flexibility in the central region to maintain the integrity of helical hairpin.

Improved Design of the Hendecad Insert:

A simple step to tighten up the targeted turn region and to improve helical propensity would be to replace the tetraglycine with tetraalanine. This works in another of our design systems (Hicks et al., Journal of Structural Biology, 137(1-2): 73-81, 2002), but still destabilises parallel dimers considerably. Therefore, the inventors chose only to replace the two outer glycines with Ala. The second position of the tetrapeptide insert is potentially a buried h site of the eleven-residue repeat (Hicks et al., Folding & Design, 2(3): 149-158, 1997; Harbury et al., Science, 282(5393): 1462-1467, 1998). Based on earlier work on peptides with consensus heptad-hendecad-heptad sequences drawn from a cytoskeleton protein from Giardia lamblia (Hicks et al., Folding & Design, 2(3): 149-158, 1997); a Lys was placed at this site. The inventors made the third position Gln to improve solubility and helix propensity. Thus, in CSP-3 the GGGG insert was replaced by AKQA. In addition, having introduced another potentially buried lysine in the helical dimer—i.e., at the new h site—the inventors replaced the Lys at the a site of the original design with Ile (see Table 1).

CD spectra of CSP-3r indicated an approximate three-fold improvement in helicity over CSP-2r (see FIG. 3A). Moreover, this structure thermally unfolded with a sigmoidal transition and a T_{M} of 40±2° C. (see FIG. 3B). The helix content and stability of CSP-3r was concentration dependent and sedimentation equilibrium analysis revealed a monomer-dimer equilibrium with a dissociation constant of 191 mM.

In its oxidised form, CSP-3o, the peptide showed marginally improved helicity and thermal stability over CSP-2o (see FIGS. 2C&D & 3A&B) and the sedimentation equilibrium analysis confirmed that CSP-3o was monomeric (see Table 2).

At this stage, having established a reasonable design sequence in which both target structures could be accessed (see FIGS. 2C&D). In particular, for CSP-3 the ΔT_{M} for the dimer and helical-hairpin forms was just 9° C. at 100 μM peptide. The inventors chose to explore the interactions that they had introduced into the designs. The starting point for these experiments was CSP-3.

The Influence of the Designed Inter-Helix Salt Bridges:

To assess the contribution of core-flanking charged residues to the thermal stability of the hairpin state, the inventors prepared control peptides in which pairings at e and g were swapped to disfavour an anti-parallel coiled coil. Recall that in the parent peptide four oppositely-charged g:e pairs were introduced to favour parallel dimer formation, and that these were made compatible with the anti-parallel helical hairpin by swapping their polarity halfway through the peptide, i.e. the g:e pairs read Glu:Lys, Glu:Lys, Lys:Glu and Lys:Glu along the sequence. To keep these compatible with the parallel dimer design, but to make potentially repulsive e:e and g:g interactions in the hairpin the inventors swapped polarity to make all the g:e pairs either Glu:Lys (CSP-4), or Lys:Glu (CSP-5) (see Table 1). Otherwise, the sequences were kept the same. Thus, the compositions of CSP-3, 4 and 5 were identical. The inventors call peptide redesigns of this type anagram mutations.

Both CSP-4o and CSP-5o behaved as monomers in AUC (see Table 2). In both cases the peptides were marginally less helical than the parent, CSP-3o (see FIG. 3A). Interestingly, however, both of the anagram mutants were thermally destabilised considerably and by roughly the same amount, ˜20° C., compared with CSP-3o. This is fully consistent with the shuffling experiment, and the design principle that the oppositely-charged e:e and g:g pairs present in CSP-3o stabilise the anti-parallel target.

The affects of the redesigned g:e interactions on the coiled-coil dimer states of CSP-4r and CSP-5r, however, were different. Both peptides formed helical dimers (see FIG. 3A and Table 2), but the thermal stability of CSP-4r was higher than CSP-3r by ˜8° C., whereas that of CSP-5r was lower than the parent by 12° C. (see FIG. 3B). Possible explanations for this 20° C. difference in the thermal stabilities of CSP-4r and CSP5r include: 1) the polarity of the g:e pairs affects local helix dipoles, or helix capping; however, one could reasonably assume that this effect would influence the anti-parallel states in a similar way, which was not seen. Alternatively, 2) Glu:Lys pairs at g:e may be energetically more favourable than corresponding Lys:Glu pairs in the parallel-dimer state. The inventors favour this second explanation as they observe similar affects in another system that they have designed to test electrostatic interactions in coiled-coil peptides (J M Shipway & D N Woolfson, unpublished).

It has been found that a systematic error was made in generating the data shown in FIG. 3B. The error resulted in the data shown in FIG. 3B to show the stability of the peptide to be 5° C. than the correct value. This error does not affect the actual meaning of the result.

Engineering the Hendecad Insert:

In an attempt to stabilise the parallel dimer, CSP-3a, we decided to replace the lysine at h with a hydrophobic residue. Although different types of residue are observed at h positions (Brown et al., Proteins-Structure Function and Genetics, 26(2): 134-145, 1996; Hicks et al., Folding & Design, 2(3): 149-158, 1997), there are very few examples of high-resolution structures for dimeric coiled coils that encompass non-canonical hendecad regions to help guide the choice of residue at h more definitively; structures include the nucleotide exchange factor GrpE (PDB identifier 1dkg, (Harrison et al. Science, 276(5311): 431-435, 1997), and the 2B region of the intermediate filament protein vimentin (PDB identifier 1gk4, (Strelkov et al., EMBO Journal, 21(6): 1255-1266, 2002). In both cases, the a-b bonds of side chains at h positions point directly towards each other. This contrasts with knobs-into-holes packing at a and d sites of canonical coiled-coil dimers, where side chains form complementary side-by-side interactions (Crick et al., Acta. Cryst., 6, 689-697, 1953; O'Shea et al., Science, 254(5031): 539-44, 1991; Harbury et al., Science, 262(5138): 1401-1407, 1993; Walshaw et al., Journal of Molecular Biology, 307(5): 1427-1450, 2001). Potentially butting side chains at h are not ideal; indeed, SOCKET analyses (Walshaw et al., Journal of Molecular Biology, 307(5): 1427-1450, 2001) of the GrpE and vimentin coiled coils indicated that the packing around the non-canonical units is considerably relaxed compared with that in canonical coiled-coil interfaces. For these reasons the inventors chose the small (and helix-favouring) residue alanine for the replacement of the h position in the CSP-3 to CSP-6 design iteration (see Table 1). Thus, all of the prescribed a, d and h positions of CSP-6 were hydrophobic. Consistent with this, the helicity and thermal stability of CSP-6r were significantly increased over the other hendecad-containing designs CSP-2r and CSP-3r, and comparable to the pure-heptad-based design CSP-1r (see FIGS. 3A&B). Importantly, CSP-6r was dimeric like the foregoing designs (see Table 2). Interestingly, however, both the helicity and stability of disulphide cross-linked form, CSP-6o, were similar to those recorded for CSP-3o. As a result the difference in thermal stability between the reduced and oxidised forms of these peptides measured at 100 μM peptide concentrations, ΔT_{M}, increased from about −10° to +22° C. in the CSP-3 to CSP-6 iteration.

In order to tune the peptides of the present invention low ΔT_{M}s between the target conformational states are required. Of course, because the reduced-and-alkylated peptides dimerise their thermal stabilities can be tuned by changing the concentration; indeed, at the lower peptide concentration of 10 μM CSP-6r remained a fully helical dimer, but with a lowered T_{M} of 61±2° C. Nonetheless, to reduce the ΔT_{M} for CSP-6 by redesign, the inventors attempted to destabilize the parallel dimer form by replacing the Ile at the a site immediately following the hendecad unit with Lys to give CSP-7 (see Table 1). As expected, this substitution compromised both the helicity and the thermal stability of CSP-7r (see FIGS. 3A&B). The stability of the oxidised form, CSP-7o, was affected less. As a result, ΔT_{M} between the two states was reduced from about +22° C. for CSP-6 to about −13° C. for CSP-7.

Study of Conformational Switching of Oxidised Form of CSP-3 to the Reduced Form

The oxidised form of CSP-3 (CSP-3o) was made up to 300 μl (100 μM) and a wavelength scan performed as before. The molar ellipticity at 222 nm was recorded. To induce the switch of the peptide from its oxidised to its reduced form a reducing agent for the disulphide bond was added. Two reducing agents were used for comparison, DTE and β-mercaptoethanol, in 20-fold excess (2 mM).

A sample of 100 μM CSP-3o plus 2 mM reducing agent was made up to a final volume of 300 μl, rapidly mixed and a time scan initiated immediately. The time taken to initiate the scan after addition of reducing agent was noted. The time scan was performed at 222 nm, 20° C., with a 4 second response time and a 10 second data pitch until the peptide was fully reduced, i.e. no further change in helicity could be seen.

A wavelength scan (190-260 nm) was run for each sample once the switch experiment was complete to record the spectrum of the reduced peptide.

The addition of excess of reducing agent to the oxidised sample caused the constraining disulphide bond to break and induced the switch from the oxidised to the reduced state. The switch reaction was studied using a time course experiment with CD spectroscopy (see FIG. 4)

The conformational switch in the presence of DTE was more rapid than that in the presence of βME. Reduction was complete with 75 minutes with DTE compared to 180 minutes with βME.

The data obtained from the time course reaction was analysed using the data analysis program Origin (v. 11) (see FIG. 5)

A single exponential curve for a first order reaction has been fitted to the data from a switch experiment, using the data analysis program Origin. The equation for the curve is:
y=Ae^{-kt}+B

where A=amplitude of signal
B=initial signal
k=rate constant
t=time

This generated a rate constant (k) for the reaction, which was then used to determine the half-life (t_{1/2}) for the reaction, using the equation:
t_{1/2}=1n2/k

This analysis was repeated for the reduction experiments with βME (see FIG. 6)

The rate of the reaction in the presence of DTE is faster than than with βME. This in turn results in a shorter half-life with DTE (see Table 3)

The results show that peptide CSP-3 can change confirmation from the oxidised form to the reduced form.

Conclusion:

Using straightforward sequence-to-structure rules and an iterative design approach the inventors have succeeded in engineering peptides with sequence and structural duality for a helical dimer and a monomeric, helical hairpin; that is, sequences in which two patterns are superimposed, one compatible with a dimeric leucine zipper and the other for an anti-parallel coiled-coil. In the specific experiments, the helical-hairpin state was favoured by cross-linking the termini with a disulphide bridge, whilst the alternative, coiled-coil dimer was favoured by keeping the terminal Cys residues reduced. The starting point in the design process was a canonical leucine-zipper template, CSP-1, which was constructed de novo based on established design rules. This peptide formed a dimeric helical structure as expected in the reduced state, but was only poorly folded in the oxidised state. In the next step, the inventors introduced a central non-heptad unit into the peptides (CSP-2 and above). This was an important feature in the success of our design process. The eleven-residue (hendecad) units allowed more flexibility for the turn in the helical hairpin, but were also compatible with the helical-dimer state. Some engineering of the hendecad/turn unit was required to optimise the structures and stabilities of the targeted motifs, which was achieved in a small number of iterations.

Comparison of the data for the later, CSP-3, 6 and 7, designs is interesting (see FIGS. 3A&B): whilst the reduced, dimeric forms displayed a broad range of thermal stabilities (the T_{M}s were 40, 75 and 34±2° C. respectively), those for the oxidised states were much tighter (T_{M}s of 50, 47 and 53±2° C. respectively). Thus, once the design was made compatible with the helical-hairpin target by introducing the hendecad insert, this structure was less sensitive to sequence changes than the alternative helical dimer. A plausible explanation for this is that in the hairpin state the hendecad acts mostly as, a turn to tether the two helical regions together, and possibly contributes little to the hydrophobic core of the structure. In the dimeric form, however, residues at the a, d and h sites of the hendecad almost certainly do contribute to the hydrophobic interface and, thus, to the overall stability of the structure. The example demonstrates that a conformation switch of the type attempted can be tuned by focusing on interactions in the dimer state. On another point, CSP-6 displays the biggest difference in thermal stability between the hairpin and dimeric forms (the latter being the more stable). Thus, with the peptide in its oxidised, hairpin state the dimeric formed might be considered as the most stressed or frustrated of all of the current designs; in other words, there is more stabilisation energy to be gained in switching from the oxidised to the reduced form than in the other designs. As the inventors have designed a redox switch with a disulphide bond as the trigger, the difference in stabilities of the oxidised and reduced states may provide a route for modulating the redox potential of the bond. Using the same design principles, tunable redox sensors for applications in cell biology may be developed (Ostergaard et al., EMBO Journal, 20(21): 5853-5862, 2001). The designs presented here may also be used to engineer conformational switches that respond to more subtle changes in solution conditions such as pH (Zhong et al., Molecular Cell, 2(1): 101-108, 1998; Skehel et al., Annual Review of Biochemistry, 69: 531-569, 2000 and Cabezon et al., EMBO Journal, 20(24): 6990-6996, 2001) and small-ligand binding (Mizoue et al., Current Opinion in Structural Biology, 12: 459-463, 2002), which potentially open routes to peptide-based sensors (Ghosh et al., Journal of the American Chemical Society, 122(23): 5658-5659, 2000).

All documents cited above are incorporated herein by reference.

TABLE 1

Sequences of CSP designs (mutations in succes-
sive iterations are highlighted in bold).

| Design | Maya's | |
|---|---|---|
| itera- | Nomen- | defgabcdefgabcdefghijkabcdef |
| tion | clature | gabcdefga |
| 1 | CSP-2 | CGGEIRALKYEIARLKQ----KKRALEQKIAA |
| | | LEGGC |
| 2 | CSP-4 | CGGEIRALKYEIAR |
| | | LKQGGGGKKRALEQKIAALEGGC |
| 3 | CSP-3 | CGGEIRALKYEIARLKQAKQA |
| | | KIRALEQKIAALEGGC |
| 4 | CSP-5 | CGGEIRALKYEIARLKQAKQAEI |
| | | RALKQEIAALKGGC |
| 5 | CSP-6 | CGGKIRALEYKIARLEQA |
| | | KQAKIRALEQKIAALEGGC |
| 6 | CSP-7 | CGGEIRALKYEIARLKQAAQA |
| | | KIRALEQKIAALEGGC |
| 7 | CSP-8 | CGGEIRALKYEIAR |
| | | LKQAAQAKKRALEQKIAALEGGC |

TABLE 2
Solution-phase molecular weights and association constants for the CSP designs.

| Design | | | | MW in solution (Da) | K_{D} (μM) |
|---|---|---|---|---|---|
| iteration | Cysteine oxidation | Maya's Nomenclature | Mᵣ | (95% confidence limits) | (95% confidence limits) |
| 1 | reduced & alkylated | CSP-2a | 3761 | 6885 (6639, 7127) | 33 (22, 48) |
| | oxidised | CSP-2o | 3643 | 3260 (3018, 3495) | — |
| 2 | reduced & alkylated | CSP-4a | 3988 | 3901 (3708, 4088) | — |
| | oxidised | CSP-4o | 3872 | 3811 (3642, 3975) | — |
| 3 | reduced & alkylated | CSP-3a | 4143 | 6652 (6372, 6925) | 191 (142, 256) |
| | oxidised | CSP-3o | 4027 | 3522 (3308, 3731) | — |
| 4 | reduced & alkylated | CSP-5a | 4143 | 6949 (6715, 7180) | 77 (57, 103) |
| | oxidised | CSP-5o | 4027 | 3768 (3563, 3968) | — |
| 5 | reduced & alkylated | CSP-6a | 4143 | 6819 (6336, 7286) | 72 (48, 106) |
| | oxidised | CSP-6o | 4027 | 3810 (3591, 4023) | — |
| 6 | reduced & alkylated | CSP-7a | 4086 | 7148 (6931, 7361) | 34 (24, 48) |
| | oxidised | CSP-7o | 3970 | 3665 (3535, 3791) | — |
| 7 | reduced & alkylated | CSP-8a | 4101 | 5481 (5199, 5755) | 506 (382, 670) |
| | oxidised | CSP-8o | 3985 | 3295 (3137, 3449) | — |

TABLE 3
Half-life of conformational switching

| Reducing | Rate (k) (sec⁻¹) | | t_{1/2} (minutes) | | |
|---|---|---|---|---|---|
| Agent | 1 | 2 | 1 | 2 | Average |
| DTE | 6.95 × 10 − 4 | 7.62 × 10 − 4 | 16.62 | 15.16 | 15.89 ± 0.73 |
| βME | 2.25 × 10 − 4 | 2.24 × 10 − 4 | 51.30 | 51.36 | 51.43 ± 0.13 |

1. A peptide having sequence and structural duality that can reversibly switch between a first structural form that can oligomerise and a second monomeric structural form in response to a stimulus, wherein the peptide comprises interactive motifs that interact to form a bond in response to the stimulus and thereby cause the peptide to assume the second structural form.

2. A peptide having sequence and structural duality which can reversibly switch between a first structural form and a second structural form in response to a stimulus, wherein the peptide comprises interactive motifs that interact to form a bond in response to the stimulus and thereby cause the peptide to assume the second structural form, wherein the peptide does not switch between an a helical structure and a β sheet structure.

3. The peptide according to claim 1 or claim 2, wherein the peptide forms a continuous helical structure as the first structural form.

4. The peptide according to claim 3, wherein the first structural form of the peptide can oligomerise.

5. The peptide according to claim 4, wherein the first structural form of the peptide dimerises to form a parallel coiled-coil dimer.

6. The peptide according to claim 1 or 2, wherein the peptide forms a hairpin structure as the second structural form.

7. The peptide according to claim 6, wherein the second structural form of the peptide is an antiparallel coiled-coil monomer.

8. The peptide according to claim 1 or 2, wherein the peptide has two different superimposed sequence and structural motifs.

9. The peptide according to claim 8, wherein the first sequence motif is a coiled-coil motif for a parallel coiled-coil dimer structure.

10. The peptide according to claim 8, wherein the second sequence motif is a coiled-coil motif for an antiparallel coiled-coil monomer structure.

11. The peptide according to claim 1 or 2, wherein the interactive motifs form a covalent or non-covalent bond in response to a stimulus.

12. The peptide according to claim 11, wherein the interactive motifs are cysteine residues which form a di-sulphide linkage in response to an oxidising environment.

13. The peptide according to claim 11, wherein the interactive motifs comprise parts of a metal binding site which form a non-covalent linkage in the presence of a corresponding metal ion.

14. The peptide according to claim 11, wherein the interactive motifs comprise parts of an antigen binding site of an antibody molecule which form a non-covalent linkage in the presence of the corresponding antigen.

15. The peptide according to claim 11, wherein the interactive motifs comprise parts of a ligand binding site of a receptor which form a non-covalent linkage in the presence of the corresponding ligand.

16. The peptide according to claim 12, having the following sequence:
X¹-(Y)ₙ-(abcdefg)ₘ-abcdef((Z)ₚ)g-(abcdefg)ₘ-abcde-(Y)ₙ-X²
wherein:
X¹ and X² are motifs capable of interacting to form a bond;
Y is any amino acid capable of acting as a linker;
ₙ is independently selected form 0 to 20;
abcdefg is a heptad sequence motif;
ₘ is 1 to 20;
abcde is the first 5 residues of a heptad sequence motif;
Z is an amino acid that is compatible with the 2 structural forms of the peptide; and
ₚ is 1 to 6.

17. The peptide according to claim 16, wherein X¹ and X² are an amino acid capable of forming a disulphide link.

18. The peptide according to claim 17, wherein X¹ and X² are cysteine.

19. The peptide according to claim 16, wherein Y is independently selected from glycine, serine or β-alanine.

20. The peptide according to any one of claims claim 16, wherein the d of the heptad sequence is leucine

21. The peptide according to claim 16, wherein the a of the heptad sequence is independently selected from isoleucine, valine, lysine, asparagine and arginine, provided a is lysine, asparagine or arginine in only 1 in every 4 heptad sequences of the peptide.

22. The peptide according to any one of claims claim 16, wherein the a of the heptad sequence is isoleucine or lysine provided a is only lysine in one of the heptad sequences of the peptide.

23. The peptide according to claim 16, wherein the g and e of the heptad repeat are oppositely charged and the polarity of the g and e are reversed in the C-terminal half of the peptide compared to the N-terminal half of the peptide.

24. The peptide according to claim 16, wherein Z is independently selected from glycine, alanine and glutamine.

25. The peptide according to claim 16, wherein (Z)ₚ is Ala-Lys-Gln-Ala; or Ala-Ala-Gln-Ala.

26. The peptide according to claim 12, wherein the peptide has the following sequence:
CGGEIRALKYEIARLKQAKQAKIRALEQKIAALEGGC;
CGGEIRALKYEIARLKQAAQAKIRALEQKIAALEGGC;
or
CGGEIRALKYEIARLKQAAQAKKRALEQKIAALEGGC.

27. (canceled)

28. A method of detecting a stimulus comprising incubating a peptide according to claims 1 or 2 with a test solution, and determining if the peptide has the first structural form or the second structural form in the test solution, wherein if the peptide assumes the second structural form, the test solution contains the stimulus.

29. A method for constructing a peptide that has sequence and structural duality, which can reversibly switch between a first and a second structural form in response to a stimulus, comprising:
(i) designing and producing a peptide using sequence rules for the first and second structural forms, wherein the sequence rules are superimposed in the peptide; and
(ii) providing interactive motifs in the peptide that form a bond in response to a stimulus, wherein the bond stabilises the second structural form, and wherein in the absence of the bond the second structural form is not stabilised and the peptide preferentially forms the first structural form.

30. The method of claim 29, for constructing a peptide wherein the peptide can reversibly switch between a first structural form that can oligomerise and a second monomeric structural form in response to a stimulus or does not switch between an α helical structure and a β sheet structure.
